(19) United States
(12) Patent Application Publication
(10) Pub. No.: US 2018/0270472 A1
(43) Pub. Date: Sep. 20, 2018

(54) DEVICE AND METHOD FOR OPTICAL ACQUISITION OF THREE-DIMENSIONAL SURFACE GEOMETRIES

(71) Applicant: a.tron3d GmbH, Klagenfurt am Woerthersee (AT)

(72) Inventor: Juergen JESENKO, Riegersburg (AT)

(21) Appl. No.: 15/917,929

(22) Filed: Mar. 12, 2018

(30) Foreign Application Priority Data
Mar. 17, 2017 (EP) 17 161 602.2

(51) Int. Cl. H04N 13/221; A61C 9/00; G08B 6/00; H04N 13/296; A61B 1/24; A61B 1/00

(52) U.S. Cl. CPC H04N 13/221; A61C 9/0053; G08B 6/00; H04N 2213/001; A61B 1/24; A61B 1/00055; H04N 13/296

(57) ABSTRACT

In a device and a method for optical acquisition of three-dimensional surface geometries with a handpiece (1), with the handpiece (1) having one or more means (2) for output of status reports, at least one means (2) for output of status reports is a means (2) for generating oscillations that outputs haptically perceptible status reports.

### BACKGROUND OF THE INVENTION

### Field of the Invention

The invention relates to a device and a method for optical acquisition of three-dimensional surface geometries with a handpiece, the handpiece having one or more means for output of status reports.

### Description of the Related Art

In dentistry, it is necessary for many tasks to have available a model of the teeth and also other intraoral structures. While, in the past these casts were produced by physical impressions and were stored physically, for example, as plaster casts, it is now more and more conventional to acquire and store these models virtually. Thus, not only can space for storing the models be reduced, but the patient is spared the unpleasant taking of the impression. Intraoral scanners, which work optically to produce virtual models of intraoral structures, have become established in the meantime and are being continuously improved. In these improvements, the focus is on not only the continuous enhancement of the precision of the acquired cast and the reduction of the required time of scanning, but also on increasing the ease of use—not only for the patient but also for the person operating the scanner or guiding a handpiece of the scanner respectively.

One important element in the improvement of user-friendliness is the communication of feedback from the scanner to the operator that is as simple as possible.

### SUMMARY OF THE INVENTION

Therefore, the object of the invention is to provide an improved possibility for providing feedback to the user of an intraoral scanner.

This object is achieved according to the invention by a device with the features of claim 1 and a method with the features of claim 3.

When virtual models of intraoral structures are being produced by the optical acquisition of their surface geometries, in addition to the actual data about the geometry of the model, various additional and useful data, for example on the status, the precision and the like, can be generated and output. In the state of the art, the output of the status and optionally of additional information and/or action instructions conventionally takes place visually, in less frequent cases even acoustically. Both methods, however, entail disadvantages for the user. Acoustic signals are often perceived as disruptive. Optical signals can divert the attention of the user.

Therefore, it is provided according to the invention that at least one means for outputting status reports is a means for generating oscillations, in particular vibrations. Thus, next to known visual and acoustic signals, additional or even exclusively haptic signals can be outputted to the user.

In this case, signals and status reports within the scope of the invention include general feedback and/or also action instructions.

In one especially preferred embodiment of the invention, the haptic signals are output when the quality of recording falls below a given threshold value. This measure may seem paradoxical even to one skilled in the art since it is intuitively assumed that the quality of optically acquired data is only further degraded when the handpiece is set into oscillation. It has been shown, however, that starting at a relatively high image rate of the scanner sensors, exactly the opposite effect can be achieved. Instead of degrading the recordings, the oscillation of the handpiece and thus also of the sensors located in it leads to an improvement of the recording.

Sensors that have been set into oscillation acquire the surface from several slightly different positions and thus create a higher density of geometric information on the acquired surface than sensors that are—compared to oscillating sensors—at rest. In this case, “at rest” compared to “oscillating” also includes the slow movement of the handpiece, which is being guided by an operator.

When the handpiece is therefore set into oscillation as the recording quality deteriorates, the haptic information about the deterioration is output not only to the operator, but at the same time a measure to eliminate the deterioration is already initiated.

Of course, it is possible to output more than only the signals “handpiece is vibrating” and “handpiece is not vibrating.”

A first possibility for varying the signals is to change the intensity of the oscillations or to generate defined sequences of oscillation and no oscillation. This possibility is therefore advantageous mainly because it is simple to implement. At the same time, however, the full potential of haptic outputs is not being used here.

While acoustic and visual signals can only be reproduced with very great technical effort such that they can be perceived three-dimensionally for an operator, as provided according to one preferred development of the invention, various means that are located spatially offset in the handpiece for producing oscillations can easily output information three-dimensionally.

The spatially offset arrangement of different means for generating oscillations in the handpiece also makes it possible, for example, to output direction information, and the user can, for example, be directed specifically to regions of the object that do not satisfy a certain data quality, yet. The information that is necessary for this purpose, where the handpiece is located at that moment relative to the object that is to be acquired, is automatically available in this case since it is being determined anyway in the case of optical methods. Operation instructions, such as “move handpiece farther” or “tilt handpiece to the left,” can be intuitively felt and understood by the operator who is holding the handpiece, by corresponding regions of the handpiece being set into oscillation.

According to one preferred embodiment of the invention, the acquisition takes place in different operation states and when the state changes, the output haptic signal changes. The different states correspond here to different operating modes, such as, for example, “normal recording” or “recording after a break-off” (recovery), i.e., after possibly unintended interruption of recording. Thus, the operator can be notified, for example, not only that scanning is now taking place with another operating mode, but also that a break-off of the scan has occurred at all.

In this case, within the scope of the invention, a change of the haptic signal can also mean the initiation or beginning or ending of the signal.

In another preferred embodiment of the invention, acquisition takes place in at least two defined physical regions, in which the handpiece can be located, and the output haptic signal changes when the region in which the handpiece is located changes. In doing so, the operator can be notified, for example, about whether the handpiece is located too close to or too far away from the object being acquired. In one exemplary, very simple implementation of this embodiment, only one region is defined in which optimum recordings can be made and leaving the region is interpreted as a change in the region in which optimum recordings are no longer possible. In one development of the embodiment, however, regions can be defined, for example, to which in addition to “not optimum,” also the properties “too close” or “too far away” are assigned. Different haptic signals for overly great or overly short distances from the object can then be delivered accordingly to the operator.

In another preferred embodiment of the method, the handpiece executes a movement during acquisition, certain properties are assigned to the movement, and the output haptic signal changes when the property of the movement changes. Thus, the operator can be, for example, notified that the handpiece is moving too quickly or too slowly.

Other preferred embodiments of the invention are the subject matter of the other dependent claims.

One embodiment of the invention that is preferred and that does not limit the protective scope of the claims is presented in detail below using the drawing.

### BRIEF DESCRIPTION OF THE DRAWINGS

FIG. 1 shows a schematically depicted handpiece with means for generating oscillations, which means are drawn symbolically.

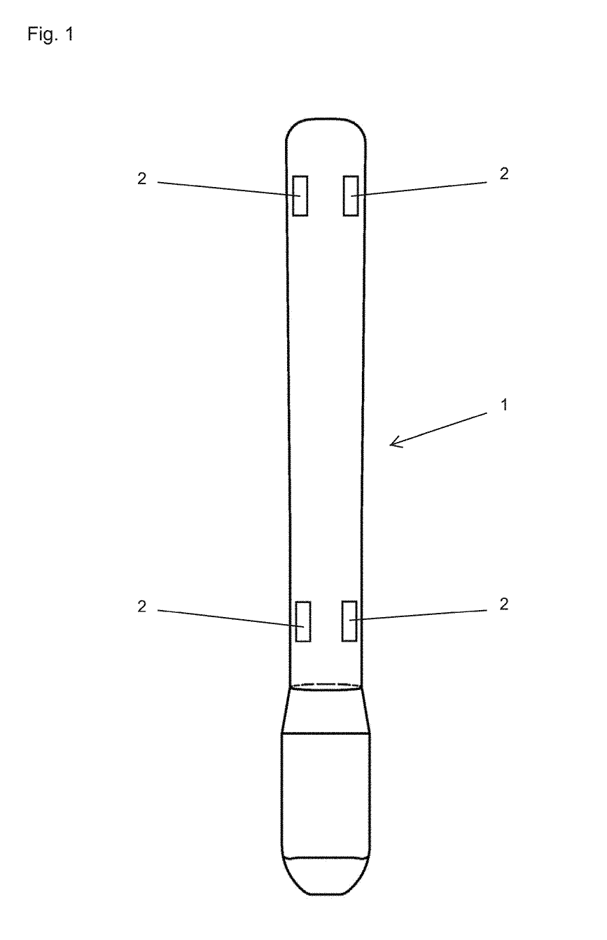

### DESCRIPTION OF THE PREFERRED EMBODIMENTS

In a schematically illustrated handpiece 1 of an intraoral scanner, four means 2 for generating oscillations are symbolically drawn. It is irrelevant to the invention which means are chosen for generating oscillations. For example, unbalance motors or even electroreactive polymers can be used.

The number of means 2 and the location at which the means 2 are mounted are likewise variable. A combination with optical and/or acoustic means for output of information, feedback, action instructions and the like is likewise possible.

1. Device for optical acquisition of three-dimensional surface geometries with a handpiece (1), the handpiece (1) having one or more means (2) for output of status reports, wherein at least one means (2) for output of status reports is a means (2) for generating oscillations.

2. Device according to claim 1, wherein in the handpiece (1), two or more means (2) for generating oscillations are arranged spatially offset, in particular diagonally to one another and/or opposite one another.

3. Method for optical acquisition of three-dimensional surface geometries with a handpiece, the handpiece (1) having one or more means (2) for output of status reports, wherein at least one means (2) outputs status reports as haptic signals by oscillations.

4. Method according to claim 3, wherein the haptic signals are output when the quality of recording exceeds or falls below a given threshold value.

5. Method according to claim 3, wherein different spatially offset means (2) for output of haptic signals oscillate with different intensity and/or staggered in time in order to output direction information.

6. Method according to claim 3, wherein acquisition takes place in different operation states and wherein when the operation state changes, the output haptic signal changes.

7. Method according to claim 3, wherein acquisition takes place in at least two defined physical regions in which the handpiece (1) can be located, and wherein the output haptic signal changes when the region in which the handpiece is located changes.

8. Method according to claim 3, wherein the handpiece executes a movement during acquisition, wherein certain properties are assigned to the movement, and wherein the output haptic signal changes when the property of the movement changes.

9. Method according to claim 3, wherein the means (2) outputs haptic signals in different intensities, frequencies and sequences.

10. Method according to claim 3, wherein acquisition takes place with an image frequency of a sensor and wherein the frequency of the haptic signal is matched to the image frequency of the sensor.

11. Method according to claim 4, wherein different spatially offset means (2) for output of haptic signals oscillate with different intensity and/or staggered in time in order to output direction information.

12. Method according to claim 4, wherein acquisition takes place in different operation states and wherein when the operation state changes, the output haptic signal changes.

13. Method according to claim 5, wherein acquisition takes place in different operation states and wherein when the operation state changes, the output haptic signal changes.

14. Method according to claim 4, wherein acquisition takes place in at least two defined physical regions in which the handpiece (1) can be located, and wherein the output haptic signal changes when the region in which the handpiece is located changes.

15. Method according to claim 5, wherein acquisition takes place in at least two defined physical regions in which the handpiece (1) can be located, and wherein the output haptic signal changes when the region in which the handpiece is located changes.

16. Method according to claim 6, wherein acquisition takes place in at least two defined physical regions in which the handpiece (1) can be located, and wherein the output haptic signal changes when the region in which the handpiece is located changes.

17. Method according to claim 4, wherein the handpiece executes a movement during acquisition, wherein certain properties are assigned to the movement, and wherein the output haptic signal changes when the property of the movement changes.

18. Method according to claim 5, wherein the handpiece executes a movement during acquisition, wherein certain properties are assigned to the movement, and wherein the output haptic signal changes when the property of the movement changes.

19. Method according to claim 6, wherein the handpiece executes a movement during acquisition, wherein certain properties are assigned to the movement, and wherein the output haptic signal changes when the property of the movement changes.

20. Method according to claim 7, wherein the handpiece executes a movement during acquisition, wherein certain properties are assigned to the movement, and wherein the output haptic signal changes when the property of the movement changes.
